(19) United States
(12) Patent Application Publication
(10) Pub. No.: US 2008/0033302 A1
(43) Pub. Date: Feb. 7, 2008

(54) SYSTEM AND METHOD FOR SEMI-AUTOMATIC AORTIC ANEURYSM ANALYSIS

(75) Inventors: Leo Grady, Yardley, PA (US); Osman Bodur, Antalya (TR); Randolph M. Setser, Shaker Heights, OH (US); Arthur E. Stillman, Chagrin Falls, OH (US); Thomas O'Donnell, New York, NY (US); Gareth Funka-Lea, Cranbury, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 11/735,529

(22) Filed: Apr. 16, 2007

Related U.S. Application Data
(60) Provisional application No. 60/793,866, filed on Apr. 21, 2006.

(51) Int. Cl. A61B 5/02

(52) U.S. Cl. 600/481; 128/898

(57) ABSTRACT

A method for automatically analyzing an aortic aneurysm includes providing a digitized 3-dimensional image volume of an aorta, determining which voxels in said image are likely to be lumen voxels, determining a distance of said lumen voxels from an aortic boundary, finding a centerline of the aorta in said image volume based on said lumen voxel distances, constructing a series of 2-dimensional multiplanar reformatted (MFR) image planes orthogonal to this centerline, segmenting aortic cross sections in each said MPR image plane wherein an aortic wall is located in each MPR image, and constructing from said aortic wall locations a 3D model of the aorta.

### CROSS REFERENCE TO RELATED UNITED STATES APPLICATIONS

This application claims priority from: “Semi-Automatic Aortic Aneurysm Analysis”, U.S. Provisional Application No. 60/793,866 of O'Donnell, et al., filed Apr. 21, 2006, the contents of which are herein incorporated by reference.

### TECHNICAL FIELD

This disclosure is directed to methods for automatically segmenting aortic cross-sections on computed tomography angiography acquisitions.

### DISCUSSION OF THE RELATED ART

The aorta is the largest artery in the body and is the primary conduit of oxygenated blood. An aortic aneurysm (AA) is a permanent and irreversible localized dilation of this vessel, and if left untreated, will gradually expand until rupture, resulting in death in 90% of cases. AAs are the 13^{th} leading cause of death in the United States. Standard procedure assesses the risk of aneurysm rupture based on maximal aortic diameter. Current clinical tools for acquiring these measurements requires a great deal of user interaction and can be quite time consuming.

Treatment for this disorder, such as open repair, runs significant risk including infection, pseudoaneurysm formation, and secondary impotence. Endovascular stent repair is gaining popularity but the long term outcome of this procedure is not yet known and not all AAs are candidates for stents. Therefore, for AAs not thought to be an imminent rupture risk, surveillance is considered preferable to immediate aggressive treatment. This is particularly true in the largest population affected by this disorder, men over 65, since morbidity from other causes may occur prior to rupture.

How to determine the rupture risk of an AA, though, is still an open question. Proposed indicators are manifold: wall stress, wall stiffness, intraluminal thrombus thickness, and wall tension have all been suggested. Standard procedure, however, calls for intervention (open repair or stent) when the maximal diameter is greater than 5.5 cm. The change over time of the maximal diameter has also been put forth as a prognostic measure.

Currently there are two common approaches to aortic diameter measurement. The first involves making linear measurements on a Maximum Intensity Projection (MIP) of the image volume. However, the selection of MIP projection angle may introduce a high degree of subjectivity in this measurement. The second employs a double oblique MPR to obtain a reconstructed image orthogonal to the vessel path on which measurements are made. The drawback of this approach is that it is time consuming and as a result the aorta may be sparsely sampled due to practical limits on the duration of the analysis. In addition, when performed manually, the orthogonal plane may not be correct, introducing error. Reproducing the same orthogonal cross-section position in a longitudinal study may also prove difficult. Finally, the manual measurement made may not be correct as it relies on the user subjectively determining which points are connected to form the maximal diameter.

One approach uses 3D level sets to segment the lumen as well as vessel border. For the vessel border a stopping criterion based on the assumption that the aortic surface is smooth and round is employed. Centerlines are created to compute orthogonal MPRs.

Another approach is an active shape model formulation in which landmarks are defined by correlating with adjacent slices rather than training data. The model is initialized manually and the two-slice model climbs one slice at a time along the aorta. The focus is on the abdominal aorta where the central axis of the vessel is approximately perpendicular to the image stack and thus does not require a centerline calculation, but does require a training set and runs the risk of degeneracy of the modes of variation since the aortic cross-sections are frequently circular.

Another approach is to segment aneurysms in the brain using a Geodesic Active Region Model combined with non-parametric region-based information. In this domain, however, the challenges come in the morphology of the vessel, the brain vascular being more detailed and complex as compared to the aorta, and the issue of thrombi is not addressed.

### SUMMARY OF THE INVENTION

Exemplary embodiments of the invention as described herein generally include methods and systems for automatically segmenting aortic cross-sections by semi-automatically determining the centerline of the aorta (lumen) and reconstructing a series of images orthogonal to this centerline. The vessel cross-sections are automatically segmented with a modified isoperimetric segmentation algorithm. Due to the challenges introduced by thrombi, calcifications, and the similarity in gray scale between the vessel wall and surrounding structures, segmentations may be user edited. From the edited segmentations a 3D model of the aorta is constructed. Finally, the two image volumes are registered for facilitating follow-up studies.

According to an embodiment of the invention, it is possible to obtain complete coverage of the aorta. It is not unusual for a practicing clinician to be constrained to only a few minutes to examine a study. Using system according to an embodiment of the invention, within seconds the clinician has available a series of optimal, reproducible, orthogonal cross-sections of the entire vessel which may be visually inspected for any irregularities. Further, the user can segment these cross-sections and have the guaranteed maximal diameter automatically computed. With the creation of a 3D model, it becomes possible to evaluate the efficacy of wall stress, wall stiffness, etc., as well as volumetric features. After the 3D model is constructed, stent planning is possible and the stage is set to compute rupture risk indicators such as wall stress (in conjunction with blood pressure readings). Finally, via registration, side by side comparison of the same aorta at different time points becomes straightforward. Since the monitoring of both AAs at risk as well as repaired aortas is commonplace, this feature can be valuable.

According to an aspect of the invention, there is provided a method for automatically analyzing an aortic aneurysm including providing a digitized 3-dimensional image volume of an aorta, wherein said image comprises a plurality of intensities defined on a 3D grid of voxels, determining which voxels in said image are likely to be lumen voxels, determining a distance of said lumen voxels from an aortic boundary, finding a centerline of the aorta in said image volume based on said lumen voxel distances, constructing a series of 2-dimensional multiplanar reformatted (MFR) image planes orthogonal to this centerline, segmenting aortic cross sections in each said MPR image plane wherein an aortic wall is located in each MPR image, and constructing from said aortic wall locations a 3D model of the aorta.

According to a further aspect of the invention, the method comprises providing two input voxels in said aorta to initialize said centerline.

According to a further aspect of the invention, one of said voxels is near the base of the aorta, and the other voxel is near the iliac bifurcation.

According to a further aspect of the invention, determining which voxels are likely to be lumen voxels comprises calculating a histogram using a Gaussian estimator on a distribution of intensities near each input voxel, and thresholding each volume voxel against a likelihood of belonging to the aortic lumen.

According to a further aspect of the invention, finding a centerline of the aorta comprises forming a path between said input voxels from those lumen voxels having a greatest distance from said aortic boundary.

According to a further aspect of the invention, the method comprises smoothing said centerline.

According to a further aspect of the invention, segmenting aortic cross sections in said MPR image planes comprises finding an image partition S, S̅ that minimizes an isoperimetric ratio of a cross section of said aorta and its boundary.

According to a further aspect of the invention, minimizing said isoperimetric ratio comprises representing said lumen intensities by a Laplacian matrix L whose entries are defined by voxels i, j by representing said lumen intensities by a Laplacian matrix L whose entries are defined by voxels i, j by

$${L}_{i , j} = \{ \begin{matrix}{d}_{i} & if i = j , \\ - w ( {e}_{ij} ) & if {e}_{ij} ∈ E , \\ 0 & otherwise .\end{matrix}$$

wherein eᵢⱼ represents an edge connecting neighboring voxels i,j, w(eᵢⱼ) is a weight for edge eᵢⱼ defined by w(eᵢⱼ)=e^{-(D}^{_{L}}^{(i)+D}^{_{T}}^{(i)-D}^{_{L}}^{(j)=D}^{_{T}}^{(j))}^{²} where D_{L} is an estimated lumen distribution, D_{T} is the estimated thrombus distribution, dᵢ is a degree of voxel i defined by summing the weights of edges connecting said voxel, and minimizing a cost function

$$g ( x ) = \frac{{x}^{T} ( L + γ U ) x}{{x}^{T} ( d + γ u )} ,$$

wherein d is a vector of voxel degrees, x is a partition indicator function defined by

$${x}_{i} = \{ \begin{matrix}0 & if voxel i ∈ \overline{S} \\ 1 & if voxel i ∈ S\end{matrix} ,$$

U indicates a Laplacian matrix with uniform weights, u represents the vector of degrees for a graph with uniform weights, and γ is a circularity parameter.

According to a further aspect of the invention, minimizing said cost function comprises selecting a node corresponding to the intersection of the centerline with the MPR as a ground voxel, v_{g}, eliminating the row/column corresponding to v_{g} to form a reduced Laplacian and degree vector L₀, d₀, solving L₀x₀=d₀ for x₀ allowing x to take on any real value, and thresholding the partition indicator x at a value that yields a partition corresponding to a lowest isoperimetric ratio.

According to a further aspect of the invention, the method comprises separating lumen and thrombus voxels from background voxels using a K-means method.

According to another aspect of the invention, there is provided a method of automatically analyzing an aortic aneurysm comprising providing a digitized 3-dimensional image volume of an aorta, wherein said image comprises a plurality of intensities defined on a 3D grid of voxels, finding a centerline of the aorta in said image volume, constructing a series of 2-dimensional multiplanar reformatted (MFR) image planes orthogonal to this centerline, separating aortic lumen and thrombus voxels from background voxels using a K-means method, segmenting aortic cross sections in each said MPR image plane by finding an image partition S, S̅ that minimizes an isoperimetric ratio of a circular cross section of said aorta and its boundary wherein an aortic wall is located in each MPR image, and constructing from said aortic wall locations a 3D model of the aorta.

According to a further aspect of the invention finding said aortic centerline includes providing two input voxels in said aorta to initialize said centerline, calculating a histogram using a Gaussian estimator on a distribution of intensities near each input voxel, thresholding each volume voxel against a likelihood of belonging to the aortic lumen wherein luimen vioxels are identified, determining a distance of said lumen voxels from an aortic boundary, and forming a path between said input voxels from those lumen voxels having a greatest distance from said aortic boundary, wherein said path forms a centerline.

According to another aspect of the invention, there is provided a program storage device readable by a computer, tangibly embodying a program of instructions executable by the computer to perform the method steps for automatically analyzing an aortic aneurysm.

### BRIEF DESCRIPTION OF THE DRAWINGS

FIGS. 1(a)-(c) illustrate successive stages of a segmentation process according to an embodiment of the invention.

FIG. 2 is a table showing sample results for evaluation of prototype according to an embodiment of the invention.

FIG. 3 depicts an aorta reconstructed from data in the table of FIG. 2, according to an embodiment of the invention.

FIG. 4 is a flowchart of a centerline computation method according to an embodiment of the invention.

FIG. 5 is a flowchart of an isoperimetric algorithm applied to image segmentation, according to an embodiment of the invention.

FIG. 6 is a block diagram of an exemplary computer system for implementing a method for automatically segmenting aortic cross-sections on computed tomography angiography acquisitions, according to an embodiment of the invention.

### DETAILED DESCRIPTION OF THE EXEMPLARY EMBODIMENTS

Exemplary embodiments of the invention as described herein generally include systems and methods for automatically segmenting aortic cross-sections on computed tomography angiography acquisitions. Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention.

As used herein, the term “image” refers to multi-dimensional data composed of discrete image elements (e.g., pixels for 2-D images and voxels for 3-D images). The image may be, for example, a medical image of a subject collected by computer tomography, magnetic resonance imaging, ultrasound, or any other medical imaging system known to one of skill in the art. The image may also be provided from non-medical contexts, such as, for example, remote sensing systems, electron microscopy, etc. Although an image can be thought of as a function from R³ to R, the methods of the inventions are not limited to such images, and can be applied, to images of any dimension, e.g. a 2-D picture or a 3-D volume. For a 2- or 3-dimensional image, the domain of the image is typically a 2- or 3-dimensional rectangular array, wherein each pixel or voxel can be addressed with reference to a set of 2 or 3 mutually orthogonal axes. The terms “digital” and “digitized” as used herein will refer to images or volumes, as appropriate, in a digital or digitized format acquired via a digital acquisition system or via conversion from an analog image.

According to an embodiment of the invention, a method for automatically segmenting aortic cross-sections determines the centerline of the aorta (lumen), reconstructing a series of images orthogonal to this centerline, and automatically segments the vessel cross-sections with a modified isoperimetric segmentation algorithm.

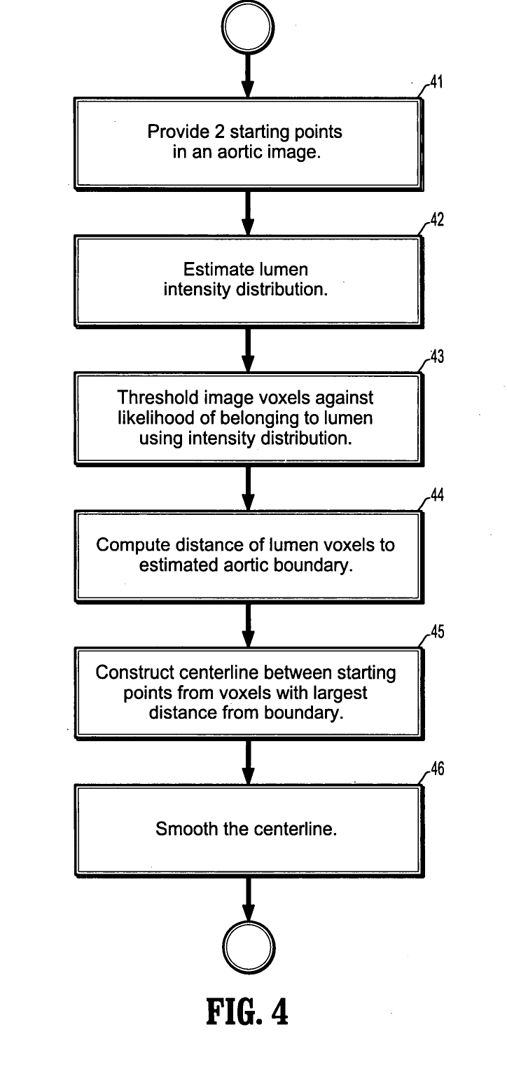

FIG. 4 is a flowchart of a centerline computation method according to an embodiment of the invention. The centerline computation is initialized by interactively providing two points at step 41, one at the base of the aorta and the other near the iliac bifurcation. From these two points, an intensity distribution of the lumen intensities is estimated at step 42 using a standard Gaussian kernel estimator on the distribution of intensities in a small neighborhood of the input points. Following the lumen intensity distribution estimation, the voxels in the volume are thresholded at step 43 against the likelihood that each voxel intensity belongs to the aortic lumen. For those voxels considered to be lumen voxels, a distance transform is computed at step 44 that estimates the distance of each lumen voxel from the aortic boundary. The aortic centerline would comprise those voxels with the highest distance values, and a path between the two input points can be constructed at step 45 from those lumen voxels having the highest distance values. This path is output as the aortic lumen centerline, which is subsequently smoothed at step 46.

The image volume is resampled at step 47 into a series of multi-planar reconstructions (MPRs) normal to the centerline. The intersection of the centerline with these images forms a point in the center of the lumen, the channel part of the aorta. This serves as input to the segmentation of the aortic border.

To determine the maximal aortic diameter, the entire vessel border is segmented. Segmentation of this border, in the presence of thrombus, which is the clotted blood in the aorta, is challenging due to the bimodal distribution of intensities within the aorta (including sharp internal boundaries) and the presence of nearby confounding structures such as the diaphragm, veins and branch vessels.

A segmentation approach according to an embodiment of the invention takes into consideration the following factors: (1) Lumen and thrombus intensities may be estimated; (2) An algorithm capable of cutting weakly-connected confounding structures should be employed; (3) The aortic cross-sections may be assumed to be generally circular; and (4) A point in the lumen from the centerline intersection is available.

To separate lumen and thrombus voxels from background voxels, a K-means algorithm is employed to cluster distinct intensity groups within the image. The K-means algorithm is an algorithm to cluster objects based on attributes into k partitions. It is a variant of the expectation-maximization algorithm in which the goal is to determine the k mean values of data generated from Gaussian distributions. The objective of the algorithm is to minimize total intra-cluster variance, or, the squared error function

$$V = {\sum }_{i = 1}^{k} {\sum }_{{x}_{j} ε {S}_{i}} {\vert {x}_{j} - {μ}_{i} \vert}^{2}$$

where there are k clusters Sᵢ, i=1, 2, . . . , k and μᵢ is the centroid or mean point of all the points xⱼεSᵢ. The algorithm starts by partitioning the input points into k initial sets, either at random or using some heuristic data. It then calculates the mean point, or centroid, of each set. It constructs a new partition by associating each point with the closest centroid. Then the centroids are recalculated for the new clusters, and algorithm repeats by alternate application of these two steps until convergence, which is obtained when the points no longer switch clusters, or alternatively, when the centroids are substantially unchanged.

According to an embodiment of the invention, the isoperimetric segmentation algorithm disclosed in “Isoperimetric Graph Partitioning for Image Segmentation”, Leo Grady and Eric L. Schwartz, IEEE Trans. on Pattern Analysis and Machine Intelligence, vol. 28, no. 3, pp. 469-475, March 2006, the contents of which are herein incorporated by reference, is a good candidate, since it takes input as a single point and can correctly cut weakly-connected confounding structures. However, this algorithm does not encourage circularity of the segmentation (on a weighted graph) and therefore requires modification.

The isoperimetric segmentation algorithm is described in terms of graph-theoretic concepts, so a description of these concepts follows. An image can be formulated as a graph G=(V, E) with voxels corresponding to vertices (nodes) vεV and edges eεE [symbol] V×V. An edge, e, spanning two vertices, vᵢ and vⱼ, is denoted by eᵢⱼ. Let n=|V| and m=|E| where | | denotes cardinality. A weighted graph has a value (typically nonnegative and real) assigned to each edge called a weight. The weight of edge eᵢⱼ, is denoted by w(eᵢⱼ) or wᵢⱼ. The degree of a vertex vᵢ, denoted dᵢ, is

$${d}_{i} {\sum }_{{e}_{ij}} w ( {e}_{ij} ) ∀ {e}_{ij} ∈ E .$$

An exemplary edge weight is defined as a function of the intensity differences of the two nodes (voxels) spanned by the edge.

The classic isoperimetric problem attempts to find, for a fixed area, the region with a minimum perimeter. More formally, the isoperimetric constant is the minimum of the ratio of the area of a boundary of a region S to its volume over all possible regions S:

$${min}_{S} \frac{\vert \partial S \vert}{{Vol}_{S}} .$$

Intuitively, a partition is sought that provides a region that is both large and has a small boundary with its surroundings. That is, it cuts weakly connected structures, such as bottlenecks. The boundary of a set, S, is defined as

$$\partial S = \{ {e}_{ij} \vert {v}_{i} ∈ S , {v}_{j} ∈ \overline{S} \} ,$$

where S̅denotes the set complement, and

$$\vert \partial S \vert = {\sum }_{{e}_{ij} ε \partial S} w ( {e}_{ij} ) .$$

The volume for a graph can be defined as

$${Vol}_{S} {\sum }_{i} {d}_{i} ∀ {v}_{i} ∈ S ,$$

where dᵢ is the vertex degree defined above. In calculating the isoperimetric ratio, regions of uniform intensity are given preference over regions possessing a large number of pixels.

The isoperimetric ratio can be expressed in matrix form. To start, define an indicator vector, x, that takes a binary value at each node:

$${x}_{i} = \{ \begin{matrix}0 & if {v}_{i} ∈ \overline{S} , \\ 1 & if {v}_{i} ∈ S .\end{matrix}$$

Note that a specification of x may be considered a partition. Define the n×n matrix, L, of a graph as

$${L}_{{v}_{i} {v}_{j}} = \{ \begin{matrix}{d}_{i} & if i = j , \\ - w ( {e}_{ij} ) & if {e}_{ij} ∈ E , \\ 0 & otherwise .\end{matrix}$$

The notation Lᵥ_{ᵢ}ᵥ_{ⱼ}, or more simply Lᵢⱼ, is used to indicate that the matrix L is being indexed by vertices vᵢ and vⱼ. This matrix is variously known as the admittance matrix or the Laplacian matrix.

Then, by definition of L, |∂S|=x^{T}Lx, and Vol_{S}=x^{T}d, where d is the vector of node degrees. Thus, the isoperimetric ratio of a graph G may be rewritten in terms of the indicator vector as

$$h ( x ) = {min}_{x} \frac{{x}^{T} Lx}{{x}^{T} d}$$

subject to the constraint that the set S has a fixed volume: Vol_{S}=x^{T}d=k. Given an indicator vector, x, h(x) represents the isoperimetric ratio associated with the partition specified by x. Segmentation of the aorta wall involves finding a partition S where S⊂V that separates the aortic epithelial layer from the surrounding tissue.

It can be shown that the solution given by the minimization of h(x), above, with uniform incident weights leads to a circle, the classical solution of the isoperimetric problem from which the algorithm was motivated. Therefore, one may combine the above term from the isoperimetric algorithm with a circularity term

$$g ( x ) = \frac{{x}^{T} ( L + γ U ) x}{{x}^{T} ( d + γ u )} ,$$

where U indicates the Laplacian matrix with uniform weights and u represents the vector of degrees for a graph with uniform weights. This minimization leads to the solution of the standard isoperimetric algorithm with the weights modified by adding a constant γ to all weights. The parameter γ controls the level of circularity enforced on the solution, with γ=0 representing no preference for circles and γ=∞ forcing the solution to be a circle, regarding of the image content. According to an embodiment of the invention, a good balance can be achieved by setting γ=0.03.

The constrained optimization of the isoperimetric ratio is made into a free variation via the introduction of a Lagrange multiplier λ and relaxation of the binary definition of x to take non-negative real values by minimizing the cost function
Q(x)=x^{T}L′x−λ(x^{T}d−k).
Since L′ is positive semi-definite and x^{T}d is nonnegative, Q(x) will be at a minimum for any critical point. Differentiating Q(x) with respect to x and setting to a minimum yields
2L′x=λd.
Thus, finding the x that minimizes Q(x) (minimal partition) reduces to solving a linear system. Henceforth, the scalar multiplier 2 and the scalar λ are dropped, since only the relative values of the solution are significant, and the prime on L will be suppressed.

Unfortunately, the matrix L is singular: all rows and columns sum to zero, so finding a unique solution to (1) requires an additional constraint.

It is assumed that the graph is connected, since the optimal partitions are clearly each connected component if the graph is disconnected (i.e., g(x)=0). Note that in general, a graph with c connected components will correspond to a matrix L with rank (n−c). If one arbitrarily designates a node, v_{g}, to include in S (i.e., fix x_{g}=0), it is reflected in (1) by removing the g^{th} row and column of L, denoted by L₀, and the g^{th} row of x and d, denoted by x₀ and d₀, so that
L₀x₀=d₀, (2)
which is a nonsingular system of equations.

Solving (2) for x₀ yields a real-valued solution that may be converted into a partition by setting a threshold. It can be shown that the partition containing the node corresponding to the removed row and column of L must be connected, for any chosen threshold, i.e., the nodes corresponding to x₀ values less than the chosen threshold form a connected component.

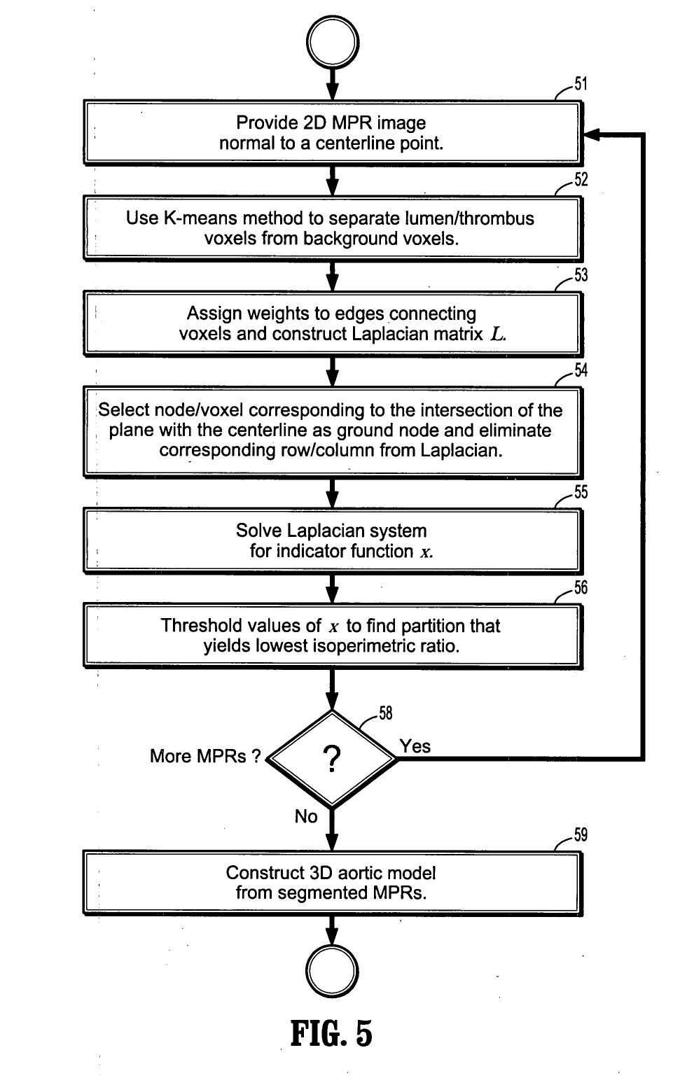

FIG. 5 is a flowchart of an isoperimetric algorithm applied to image segmentation, according to an embodiment of the invention. Referring to the figure, the algorithm begins by providing at step 51a 2D MPR image normal to the centerline.

At step 52, the K-means algorithm is applied to separate the lumen and thrombus voxels from background voxels. An exemplary, non-limiting value of K is 5. The mean corresponding to the lumen intensity is known from the location of the centerline point, and the thrombus mean is selected, by looking for a mean that is nearby to the centerline, but not belonging to the lumen mean. A mean is rejected as not representing a thrombus if the number of voxels belonging to the mean is too small or falls outside a learned range of plausible thrombus intensities.

At step 53, weights (affinities) between neighboring pixels, i and j, are defined by
wᵢⱼ=e^{-(D}^{_{L}}^{(i)+D}^{_{T}}^{(i)-D}^{_{L}}^{(j)-D}^{_{T}}^{(j))}^{²}+γ. (2)
where D_{L} is an estimated lumen distribution, D_{T} is the estimated thrombus distribution, and γ is the circularity defined above, and the L matrix is built from the weights. Note that edges connecting a lumen or thrombus vertex with a non-lumen or non-thrombus vertex are given a low weight.

At step 54, the ground node is chosen as the point on the centerline that intersects with the slice, and the corresponding row and column is eliminated from the Laplacian to determine L₀ and d₀. The equation L₀x₀=d₀ is solved for x₀ at step 55.

At step 56, the potentials x are thresholded at the value that gives partitions corresponding to the lowest isoperimetric ratio. At step 58 the algorithm loops back to repeat steps 51-56 for remaining MPRs. Finally, at step 59, a 3D model of the aorta is constructed from the sequence of segmented MPRs.

The binary definition of x can be extended to the real numbers in order to solve L₀x₀=d₀ in step 55. Therefore, to convert the solution, x, to a partition, step 56 is performed. Conversion of a potential vector to a partition may be accomplished using a threshold. A cut value is a value, α, such that S={vᵢ|xᵢ≦α} and S̅={vᵢ|xᵢ>α}. The partitioning of S and S̅ in this way may be referred to as a cut. This thresholding operation creates a partition from the potential vector, x. Note that a connected graph corresponds to an L₀ that is monotone, thus L₀⁻¹≧0. This result then implies that x₀=L₀⁻¹d₀≧0. Then, choose a threshold such that the resulting partitions have the lowest available isoperimetric ratio (the ratio cut).

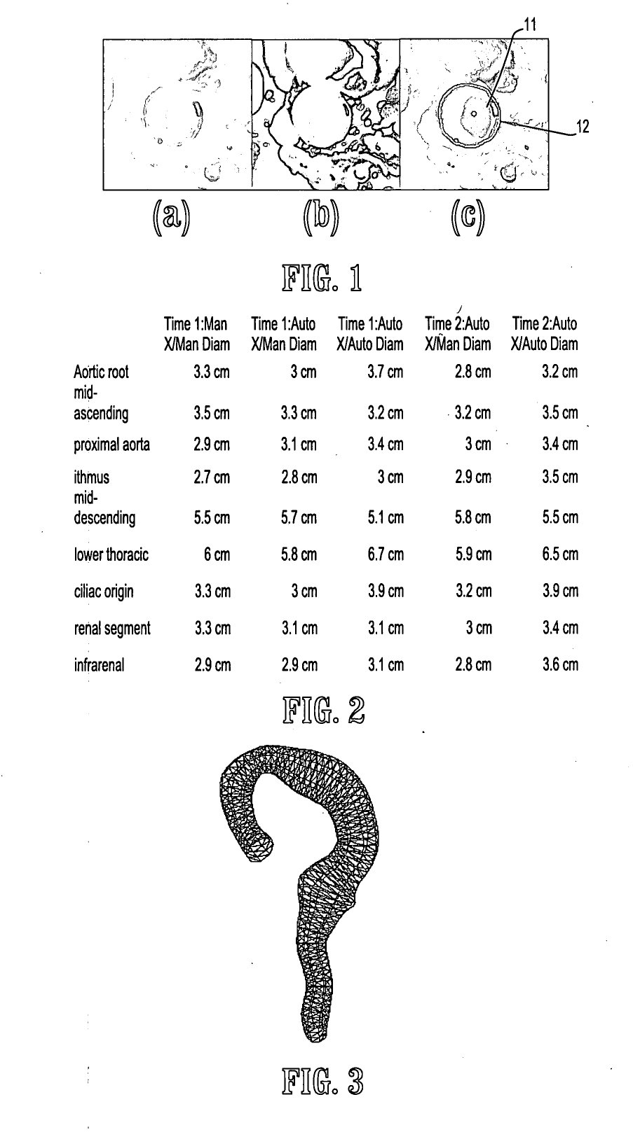

FIGS. 1(a)-(c) illustrate successive stages of a segmentation process. The left image, FIG. 1(a), shows the input image, a cross-sectional image of an aorta exhibiting thrombosis. The center image, FIG. 1(b), shows the (combined) probability map that the pixels belong to the aorta (upon which the weights are based, i.e., this is the image passed to the modified isoperimetric algorithm). The rightmost image, FIG. 1(c), displays the segmentation of the image with initial centerpoint included, where the spot 11 indicates the passed in location of the centerline and the ring 12 indicates the location of the resulting segmentation border. Note that the rightmost image has been whitened to enhance contrast with the segmentation.

An approach according to an embodiment of the invention was validated on four patients with two image volumes each (Time1 and Time2) acquired six months to 1.5 years apart. Patients were imaged at least twice between February 2002 through December 2005 using 4-slice (Volume Zoom, Siemens Medical Solutions), 16-slice (Sensation 16, Siemens) and/or a 64-slice (Sensation 64, Siemens) CT system. A contrast-enhanced, non-gated spiral examination was typically used to image the thoracic aorta in a single breathhold. Non-overlapping, 3 mm thick slices were reconstructed.

An expert radiologist reformatted the Time1 datasets manually (via double oblique) to obtain cross-sections, and measured aortic diameters manually using virtual calipers at 9 points along the aorta. Both the Time1 and Time2 datasets were loaded into a prototype according to an embodiment of the invention, which registered them and created a centerline. The expert scrolled thru the automatically generated cross-sections to approximately the same points as before and manually measured diameters as well as generating automatic diameters on both time points. An exemplary dataset for one patient is shown in the table depicted in FIG. 2. A 3D model of an aorta constructed from this patient using this data is shown in FIG. 3.

Referring now to FIG. 2, proceeding from left to right, the first column, labeled “Man X/Man Diam”, displays the Manual Cross-section\Manual Diameter Measurement on the Time 1 image volume. The second column, labeled “Auto X/Man Diam”, displays the Automatic Cross-section\Manual Diameter Measurement on the Time 1 image volume. The third column, labeled “Auto X/Auto Diam”, displays the Auto Cross-section\Auto Diameter Measurement on the Time 1 image volume. The last two columns are similarly labeled.

The average difference between Man X/Man Diam and Auto X/Man Diam for Time 1 over all patients was 0.197+/−0.152 cm. The signed difference was 0.136+/−0.209 cm with the Man X/Man Diam on average larger. This indicates that an automatic centerline method of an embodiment of the invention was on average better at finding the orthogonal image plane to the vessel. The measurement of the vessel diameter is never smaller than in the true orthogonal cross-section. The difference over all image volumes for Auto X\Man Diam and Auto X\Auto Diam was 0.342+/−0.245 cm. Each image required 0.52 edits on average.

An approach according to an embodiment of the invention can yield time savings. On average, it took the expert radiologist approximately 15 minutes to perform the manual double oblique sampling of a single dataset. With a prototype according to an embodiment of the invention, he was able to visually analyze and make measurements on two image acquisitions from the same patient in 10 minutes, and with complete coverage of the aorta.

The points chosen for comparison by the expert radiologist were often located near bifurcations of the aorta. These bifurcation points make it easier to consistently compare the same location when performing a manual analysis but make the automatic segmentation trickier since the segmentation can bleed into the adjoining vessel.

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

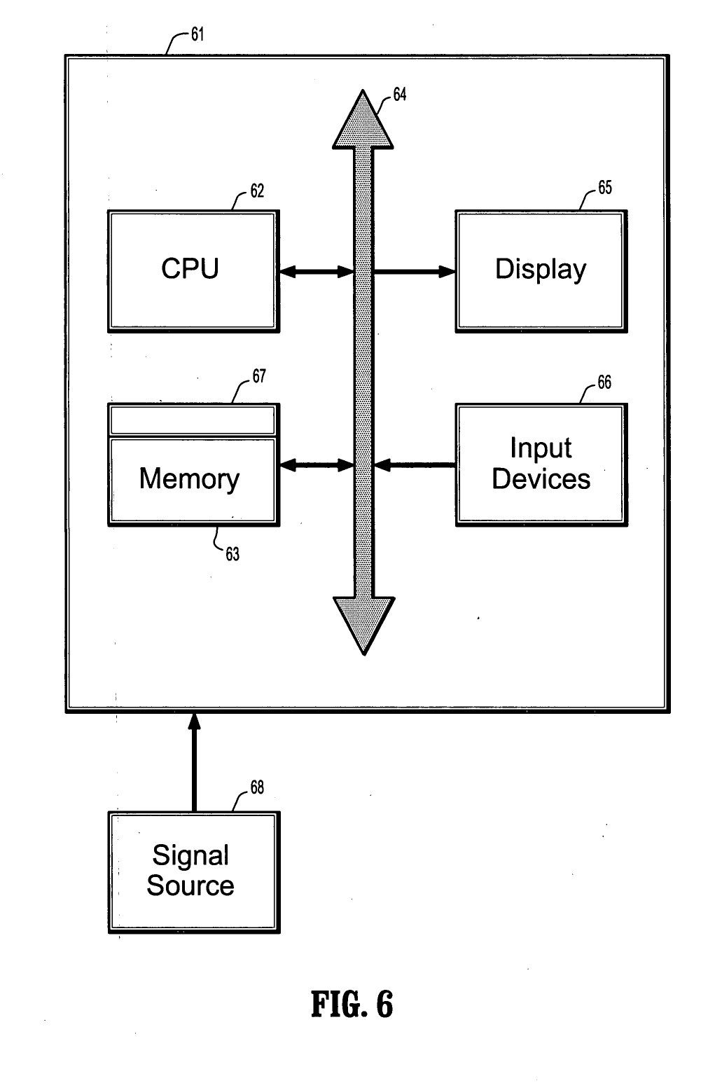

FIG. 6 is a block diagram of an exemplary computer system for implementing a method for automatically segmenting aortic cross-sections on computed tomography angiography acquisitions, according to an embodiment of the invention. Referring now to FIG. 6, a computer system 61 for implementing the present invention can comprise, inter alia, a central processing unit (CPU) 62, a memory 63 and an input/output (I/O) interface 64. The computer system 61 is generally coupled through the I/O interface 64 to a display 65 and various input devices 66 such as a mouse and a keyboard. The support circuits can include circuits such as cache, power supplies, clock circuits, and a communication bus. The memory 63 can include random access memory (RAM), read only memory (ROM), disk drive, tape drive, etc., or a combinations thereof. The present invention can be implemented as a routine 67 that is stored in memory 63 and executed by the CPU 62 to process the signal from the signal source 68. As such, the computer system 61 is a general purpose computer system that becomes a specific purpose computer system when executing the routine 67 of the present invention.

The computer system 61 also includes an operating system and micro instruction code. The various processes and functions described herein can either be part of the micro instruction code or part of the application program (or combination thereof) which is executed via the operating system. In addition, various other peripheral devices can be connected to the computer platform such as an additional data storage device and a printing device.

It is to be further understood that, because some of the constituent system components and method steps depicted in the accompanying figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

While the present invention has been described in detail with reference to a preferred embodiment, those skilled in the art will appreciate that various modifications and substitutions can be made thereto without departing from the spirit and scope of the invention as set forth in the appended claims.

What is claimed is:

1. A method of automatically analyzing an aortic aneurysm comprising the steps of:
providing a digitized 3-dimensional image volume of an aorta, wherein said image comprises a plurality of intensities defined on a 3D grid of voxels;
determining which voxels in said image are likely to be lumen voxels;
determining a distance of said lumen voxels from an aortic boundary;
finding a centerline of the aorta in said image volume based on said lumen voxel distances;
constructing a series of 2-dimensional multiplanar reformatted (MFR) image planes orthogonal to this centerline;
segmenting aortic cross sections in each said MPR image plane wherein an aortic wall is located in each MPR image; and
constructing from said aortic wall locations a 3D model of the aorta.

2. The method of claim 1, further comprising providing two input voxels in said aorta to initialize said centerline.

3. The method of claim 2, wherein one of said voxels is near the base of the aorta, and the other voxel is near the iliac bifurcation.

4. The method of claim 2, wherein determining which voxels are likely to be lumen voxels comprises calculating a histogram using a Gaussian estimator on a distribution of intensities near each input voxel, and thresholding each volume voxel against a likelihood of belonging to the aortic lumen.

5. The method of claim 2, wherein finding a centerline of the aorta comprises forming a path between said input voxels from those lumen voxels having a greatest distance from said aortic boundary.

6. The method of claim 1, further comprising smoothing said centerline.

7. The method of claim 1, wherein segmenting aortic cross sections in said MPR image planes comprises finding an image partition S, S̅ that minimizes an isoperimetric ratio of a cross section of said aorta and its boundary.

8. The method of claim 7, wherein minimizing said isoperimetric ratio comprises representing said lumen intensities by a Laplacian matrix L whose entries are defined by voxels i, j by representing said lumen intensities by a Laplacian matrix L whose entries are defined by voxels i, j by
$${L}_{i , j} = \{ \begin{matrix}{d}_{i} & if i = j , \\ - w ( {e}_{ij} ) & if {e}_{ij} ∈ E , \\ 0 & otherwise .\end{matrix}$$
wherein eᵢⱼ represents an edge connecting neighboring voxels ij, w(eᵢⱼ) is a weight for edge eᵢⱼ defined by w(eᵢⱼ)=e^{-(D}^{_{L}}^{(i)+D}^{_{T}}^{(i)-D}^{_{L}}^{(j)-D}^{_{T}}^{(j))}^{²} where D_{L} is an estimated lumen distribution, D_{T} is the estimated thrombus distribution, dᵢ is a degree of voxel i defined by summing the weights of edges connecting said voxel, and minimizing a cost function
$$g ( x ) = \frac{{x}^{T} ( L + γ U ) x}{{x}^{T} ( d + γ u )} ,$$
wherein d is a vector of voxel degrees, x is a partition indicator function defined by
$${x}_{i} = \{ \begin{matrix}0 & if voxel i ∈ \overline{S} \\ 1 & if voxel i ∈ S\end{matrix} ,$$
U indicates a Laplacian matrix with uniform weights, u represents the vector of degrees for a graph with uniform weights, and γ is a circularity parameter.

9. The method of claim 8, wherein minimizing said cost function comprises selecting a node corresponding to the intersection of the centerline with the MPR as a ground voxel, v_{g}, eliminating the row/column corresponding to v_{g} to form a reduced Laplacian and degree vector L₀, d₀, solving L₀x₀=d₀ for x₀ allowing x to take on any real value, and thresholding the partition indicator x at a value that yields a partition corresponding to a lowest isoperimetric ratio.

10. The method of claim 1, further comprising separating lumen and thrombus voxels from background voxels using a K-means method.

11. A method of automatically analyzing an aortic aneurysm comprising the steps of:
providing a digitized 3-dimensional image volume of an aorta, wherein said image comprises a plurality of intensities defined on a 3D grid of voxels;
finding a centerline of the aorta in said image volume;
constructing a series of 2-dimensional multiplanar reformatted (MFR) image planes orthogonal to this centerline;
separating aortic lumen and thrombus voxels from background voxels using a K-means method;
segmenting aortic cross sections in each said MPR image plane by finding an image partition S, S̅ that minimizes an isoperimetric ratio of a circular cross section of said aorta and its boundary wherein an aortic wall is located in each MPR image; and
constructing from said aortic wall locations a 3D model of the aorta.

12. The method of claim 11, wherein finding said aortic centerline comprises
providing two input voxels in said aorta to initialize said centerline,
calculating a histogram using a Gaussian estimator on a distribution of intensities near each input voxel;
thresholding each volume voxel against a likelihood of belonging to the aortic lumen wherein luimen vioxels are identified;
determining a distance of said lumen voxels from an aortic boundary; and
forming a path between said input voxels from those lumen voxels having a greatest distance from said aortic boundary, wherein said path forms a centerline.

13. A program storage device readable by a computer, tangibly embodying a program of instructions executable by the computer to perform the method steps for automatically analyzing an aortic aneurysm comprising the steps of:
providing a digitized 3-dimensional image volume of an aorta, wherein said image comprises a plurality of intensities defined on a 3D grid of voxels;
determining which voxels in said image are likely to be lumen voxels;
determining a distance of said lumen voxels from an aortic boundary;
finding a centerline of the aorta in said image volume based on said lumen voxel distances;
constructing a series of 2-dimensional multiplanar reformatted (MFR) image planes orthogonal to this centerline;
segmenting aortic cross sections in each said MPR image plane wherein an aortic wall is located in each MPR image; and
constructing from said aortic wall locations a 3D model of the aorta.

14. The computer readable program storage device of claim 13, the method further comprising providing two input voxels in said aorta to initialize said centerline.

15. The computer readable program storage device of claim 14, wherein one of said voxels is near the base of the aorta, and the other voxel is near the iliac bifurcation.

16. The computer readable program storage device of claim 14, wherein determining which voxels are likely to be lumen voxels comprises calculating a histogram using a Gaussian estimator on a distribution of intensities near each input voxel, and thresholding each volume voxel against a likelihood of belonging to the aortic lumen.

17. The computer readable program storage device of claim 14, wherein finding a centerline of the aorta comprises forming a path between said input voxels from those lumen voxels having a greatest distance from said aortic boundary.

18. The computer readable program storage device of claim 13, the method further comprising smoothing said centerline.

19. The computer readable program storage device of claim 13, wherein segmenting aortic cross sections in said MPR image planes comprises finding an image partition S, S̅ that minimizes an isoperimetric ratio of a cross section of said aorta and its boundary.

20. The computer readable program storage device of claim 19, wherein minimizing said isoperimetric ratio comprises representing said lumen intensities by a Laplacian matrix L whose entries are defined by voxels i, j by representing said lumen intensities by a Laplacian matrix L whose entries are defined by voxels i, j by
$${L}_{i , j} = \{ \begin{matrix}{d}_{i} & if i = j , \\ - w ( {e}_{ij} ) & if {e}_{ij} ∈ E , \\ 0 & otherwise .\end{matrix}$$
wherein eᵢⱼ represents an edge connecting neighboring voxels i,j, w(eᵢⱼ) is a weight for edge eᵢⱼ defined by w(eᵢⱼ)=e^{-(D}^{_{L}}^{(i)+D}^{_{T}}^{(i)-D}^{_{L}}^{(j)-D}^{_{T}}^{(j))}^{²} where D_{L} is an estimated lumen distribution, D_{T} is the estimated thrombus distribution, dᵢ is a degree of voxel i defined by summing the weights of edges connecting said voxel, and minimizing a cost function
$$g ( x ) = \frac{{x}^{T} ( L + γ U ) x}{{x}^{T} ( d + γ u )} ,$$
wherein d is a vector of voxel degrees, x is a partition indicator function defined by
$${x}_{i} = \{ \begin{matrix}0 & if voxel i ∈ \overline{S} \\ 1 & if voxel i ∈ S\end{matrix} ,$$
U indicates a Laplacian matrix with uniform weights, u represents the vector of degrees for a graph with uniform weights, and γ is a circularity parameter.

21. The computer readable program storage device of claim 20, wherein minimizing said cost function comprises selecting a node corresponding to the intersection of the centerline with the MPR as a ground voxel, v_{g}, eliminating the row/column corresponding to v_{g} to form a reduced Laplacian and degree vector L₀, d₀, solving L₀x₀=d₀ for x₀ allowing x to take on any real value, and thresholding the partition indicator x at a value that yields a partition corresponding to a lowest isoperimetric ratio.

22. The computer readable program storage device of claim 13, the method further comprising separating lumen and thrombus voxels from background voxels using a K-means method.
